(19) United States
(12) Patent Application Publication
(10) Pub. No.: US 2026/0325470 A1
(43) Pub. Date: Oct. 22, 2026

(54) Rapid Onset Therapeutic Ketosis Enabled by D-beta-hydroxybutyric acid Systems, Kits and Methods for Sepsis Protection and Enhanced Medical Recovery

(71) Applicant: Global Ketone Limited, Hong Kong SAR (HK)

(72) Inventors: Adrian Soto-Mota, Mexico City (MX); Heidi Heather Hynes, Seattle, WA (US)

(21) Appl. No.: 19/645,156

(22) Filed: Apr. 11, 2026

Related U.S. Application Data
(63) Continuation-in-part of application No. 18/401,445, filed on Dec. 30, 2023.
(60) Provisional application No. 63/478,142, filed on Dec. 31, 2022.

(51) Int. Cl. A61K 31/19; A61K 45/06

(52) U.S. Cl. CPC A61K 31/19; A61K 45/06

(57) ABSTRACT

Methods, kits, and adjunctive therapeutic regimens for inducing exogenous ketosis in critically ill patients with sepsis or septic shock using bioidentical free D-beta-hydroxybutyric acid—a salt-free, alcohol-free, ester-free formulation chemically identical to the endogenous ketone body—are disclosed. In a randomized, blinded, placebo-controlled clinical trial, daily enteral administration of approximately 10 g of bioidentical D-beta-hydroxybutyric acid for seven days in adult ICU sepsis patients caused no supplementation-attributable metabolic acidosis or clinically significant electrolyte abnormalities. Disclosed methods provide weight-based dosing frameworks, biomarker-guided dosing coefficient adjustment using inflammatory and metabolic indicators including C-reactive protein, TG/HDL ratio, glycated haemoglobin, leukocyte differentials, and predefined safety monitoring with stopping criteria. In addition, administration kits (1601) with clinician instructions for safety-focused and efficacy-focused regimens are disclosed. These findings establish a safety and tolerability foundation supporting bioidentical free D-beta-hydroxybutyric acid as an adjunctive therapy for sepsis and septic shock in the intensive care setting.

### CROSS-REFERENCE TO RELATED APPLICATIONS

This application is a continuation-in-part of U.S. Non-Provisional application Ser. No. 18/401,445, filed Dec. 30, 2023, which claims the benefit of U.S. Provisional Application Ser. No. 63/478,142, filed Dec. 31, 2022, pursuant to 35 U.S.C. § 120 and § 119 (e), respectively. The disclosures of the prior applications are incorporated herein by reference in their entirety.

### TECHNICAL FIELD

The claimed invention has applicability in hospital recovery medication. With greater particularity, the claimed invention is relevant to therapeutic interventions for sepsis. With still greater particularity, the claimed invention is directed at novel uses of the ketone body D-beta-hydroxybutyric acid for treatment of sepsis together with and independently of pharmaceutical sepsis medication with related methods and kits. In particular, it concerns methods and compositions for inducing exogenous ketosis using bioidentical D-beta-hydroxybutyric acid (also described as ‘free D-BHB’ herein) in patients with sepsis, including critically ill patients admitted to an intensive care unit, without inducing or exacerbating metabolic acidosis or electrolyte disturbances.

### BACKGROUND OF THE INVENTION

Sepsis is a life threatening organ dysfunction caused by a dysregulated host response to infection and remains a leading cause of mortality in intensive care units worldwide, with millions of deaths annually and reported mortality rates of approximately 30-50% despite advances in supportive care. Sepsis pathophysiology involves excessive inflammation, immune dysregulation, tissue damage, organ dysfunction, and profound metabolic derangements including glucose dysregulation and hyperinflammation. The inflammatory response in sepsis promotes excessive gluconeogenesis, resulting in hyperglycemia, insulin resistance, and muscle wasting; these derangements are associated with worse outcomes and underpin current recommendations for carbohydrate based enteral nutrition as the primary energy source in critically ill patients. However, emerging evidence suggests that high carbohydrate intake may exacerbate inflammatory responses and contribute to immune dysfunction in sepsis, prompting interest in alternative metabolic substrates.

Latest research published in The Lancet has found that sepsis is responsible for the most deaths worldwide, even more than cancer or coronary disease-previously believed to be the leading causes of death globally. Sepsis occurs when chemicals are released in the bloodstream to fight an infection triggering inflammation throughout the body. This can cause a cascade of changes that damage multiple organ systems, leading them to fail, sometimes even resulting in death. While treatment includes antibiotics and intravenous fluids, these often treat the symptoms and do not address or mitigate the subsequent inflammation from sepsis. Individuals undergoing a post-surgical hospital stay are at particular risk of contracting sepsis. Cardiac bypass is the most common type of heart surgery performed on adults in the US. The average cost of bypass surgery in the US in 2018 was $123,000. With an average hospital stay of 6-7 days for bypass surgery, each and every day of shortened hospital stay not only improves recovery outcomes but also reduces the likelihood of return visits due to complications. With respect to cancer surgery hospitalizations, room for improvement exists over current therapeutic approaches.

### BRIEF SUMMARY OF THE INVENTION

In sepsis, underlying metabolic acidosis and electrolyte imbalance raise concern that exogenous ketosis could worsen acid-base status, and exogenous ketones can transiently alter blood acidity and electrolytes, creating a perceived risk of inducing or exacerbating metabolic acidosis in critically ill patients. Prior to the present invention, the safety and tolerability of orally or enterally administered bioidentical D-beta-hydroxybutyric acid in septic, critically ill patients particularly with respect to acid-base homeostasis and electrolytes-had not been systematically established. The ketone body beta hydroxybutyrate (D-BHB) has received increasing attention as both an energy substrate and a signaling metabolite with pleiotropic effects on metabolism and immunity, including provision of a substantial fraction of cerebral energy under ketogenic conditions and inhibition of the NLRP3 inflammasome at physiologically achievable concentrations.

Additional signaling activities of D-BHB relevant to sepsis include inhibition of histone deacetylases that regulate glucose metabolism, activation of hydroxycarboxylic acid receptor 2 to reduce lipolysis and promote anti-inflammatory effects, and activation of free fatty acid receptor 3 to suppress pro inflammatory monocyte profiles. Clinical work in critical illness has shown that D-BHB infusion can reduce lipolysis and hepatic glucose production without adverse effects, and that ketogenic nutrition in sepsis can improve glucose-insulin metabolism and inflammatory markers and increase organ support free days, supporting a therapeutic role for ketone bodies. Nevertheless, common prior approaches to inducing ketosis have limitations: ketone salts increase mineral load, ketone esters rely on alcohol vehicles and have poor palatability, and medium chain triglycerides require carbohydrate restriction and, therefore, have a slower onset and the ratio of D-BHB they produce per dose is inconsistent; in contrast, bioidentical free D-BHB avoids these issues and has shown a favourable safety and tolerability profile in healthy adults. Moreover, recent work by Karagiannis and colleagues provides additional mechanistic insight: in COVID-19 acute respiratory distress syndrome, hepatic ketogenesis is impaired, leading to reduced circulating BHB levels and compromised CD4+ T cell function. That work demonstrates that BHB promotes T cell survival and interferon-gamma production through enhanced oxidative phosphorylation, and ketogenic diet supplementation reduced mortality in SARS-COV-2-infected mice, suggesting that exogenous ketone provision may be particularly beneficial when endogenous ketogenesis is compromised. While this mechanism was characterized in a murine model, the present disclosure—representing the first clinical trial of bioidentical free D-BHB in critically ill sepsis patients-provides the foundational human safety data from which these immunomodulatory implications may be further investigated.

The claimed invention provides methods for safely inducing exogenous ketosis in patients with sepsis, including critically ill patients in intensive care, by administering a therapeutically effective amount of bioidentical free beta hydroxybutyrate via oral or enteral routes while maintaining acid-base and electrolyte balance. The inventors have demonstrated in a randomized, blinded, placebo controlled clinical study that daily administration of free D BHB to septic patients does not cause supplementation attributable metabolic acidosis and does not significantly alter arterial pH, pCO2, bicarbonate, or serum electrolytes compared with placebo under standard critical care management. In some embodiments, the method comprises administering approximately 10 g of bioidentical D BHB per day for a period of about seven days to adult patients meeting sepsis criteria, delivered orally or via a nasogastric tube, with timing coordinated to routine blood gas assessments. Under these conditions, mean arterial pH, pCO2, and bicarbonate values remain comparable to those in placebo treated patients, and no cases of supplementation attributable metabolic acidosis or clinically relevant electrolyte derangements are observed, thereby establishing a safety profile suitable for further efficacy studies and therapeutic use. The claimed invention further provides dosing regimens, monitoring strategies, and stopping criteria that enable clinicians to administer exogenous free D-BHB in sepsis with predefined safeguards against acid-base complications. In some embodiments, the methods are implemented as adjunctive therapy to standard sepsis management, and are expected to permit the exploration of metabolic and immunomodulatory benefits of exogenous ketosis without introducing additional acid-base risk in this vulnerable population.

Furthermore, experimental evidence suggests ketosis could improve many of the coexisting pathophysiological processes sepsis patients have such as: inflammation, muscle mass loss, hyperglycemia, decreased myocardial contractility and some pathogen specific immune response pathways like those against SARS-Cov-2 and Pseudomonas. Ketosis may be naturally induced through strict diet control or fasting, neither option is a practical solution in a hospital setting for induction of rapid ketosis. While a variety of artificial exogenous ketone supplementation has been known and tried in a variety of forms over the past fifty years, both the chemically created ‘ketone salt’ and synthetic ‘ketone ester’ are non-natural solutions with direct limitations and sub-optimal consequences when deployed in a hospital setting or when used for patient recovery. Ketone chirality matters, and the often “D+L” ketone salts exhibit high salt load coupled with impaired ketosis levels. Moreover, the synthetic ‘ketone ester’ is an alcohol, an artificial, never before seen in man engineered construction of organic chemistry which requires subsequent stomach and liver processing before becoming bioavailable. It is also worth emphasizing that contrary to artificial ketone salts or synthetic esters and alcohols including 1,3 Butanediol, these are all chemical precursors which require stomach or liver processing to release their ketone moiety. Only exogenous bioidentical D-beta-hydroxybutyric acid is immediately bioavailable and requires no stomach release and has no liver impact. Consequently, there has been a long felt need and compelling reason to utilize bioidentical D-beta-hydroxybutyric acid for sepsis mitigation and hospital recovery scenarios. Applicant's exogenous D-betahydroxybutyric acid administered in the absence of a chemical salt or ester has already been the subject of ClinicalTrials.gov identifier NCT05584371. This is the first time the ‘bioidentical’ form of D-beta-hydroxybutyric acid in the absence of a ‘ketone salt’ or ‘ketone ester’ has been tested in a hospital setting with positive results. This application builds on these initial efforts with new clinical trial data specifically from the hospital ICU patient care setting.

Bioidentical D-beta-hydroxybutyric acid creates direct cardiac energy & anti-inflammation resulting in improved therapeutic outcomes. In general, bioidentical free D-beta hydroxybutyric acid (D-BHB) reduces inflammation, improves atherosclerosis along with improving cardiac energy and reducing glucose or mitigating the glucose spike from carbohydrate consumption. All of these attributes increase opportunities for improved hospitalization outcomes alone or when combined with dietary carbohydrate restriction. Bioidentical free D-beta-hydroxybutyric acid is superior to commonly known ketone salts and ketone esters for a number of reasons, not limited to its rapid bioavailability and absence of chemical salt or ester bond. Free D-BHB acid also has an opportunity for surgical recovery improvement in the absence of co-administered pharmaceuticals as well. D-BHB is direct mitochondrial cardiac, brain and kidney energy & anti-inflammation which synergistically creates enhanced recovery owing to improved mitochondrial bioenergenics during surgical recovery. With enhanced mitochondrial energy, cardiac tissue at risk of dying improve opportunities for recovery for the cardiac cell being challenged, for the cardiac tissue at risk of impairment and for the individual in terms of healthy outcome vs impairment or death.

The ketogenic diet and more recently administration of ketogenic compounds have had a similar long history with proven benefits for conditions such as epilepsy. Dietary interventions for ketogenic diet treatment of atherosclerosis are also yielding compelling results. The administration of the natural form of D-beta-hydroxybutyric acid as an exogenous compound has rapid onset and direct benefits to cardiac bioenergenics and blood circulation dynamics as well as reduced inflammation including the NLRP3 pathway. Preliminary data indicates that individuals suffering from atherosclerosis often benefit from administration of D-beta-hydroxybutyric acid in the absence of a salt or ester.

The claimed invention utilizes D-beta-hydroxybutyric acid for hospital recovery intervention as a restorative delivery system for improved physiological outcomes in the treatment of cardiac related physiological conditions among others. The exogenous ketone D-BHB Acid is either co-administered with standard pharmaceutical compounds, prophylactically or subsequently administered with the following expected benefits:

Hospital stay time is reduced and sepsis and septic shock are avoided owing to the anti-inflammatory characteristics of natural D-beta-hydroxybutyric acid. With the introduction of D-BHB Acid improving cardiac bioenergenics and reducing cardiac inflammation, it is a foreseeable consequence of the claimed invention that a shorter hospital stay after cardiac surgery will be attained leading to a higher percentage of positive physiological recovery breakthroughs.

The bioidentical form of D-beta-hydroxybutyric acid is rapidly absorbed and immediately bioavailable which is in direct contrast to the moderately ketogenic ketone salts and ketone ester compounds which require stomach disassociation followed by liver processing. The greatly improved bioavailability of free D-beta-hydroxybutyric acid improves therapeutic outcomes for hospital recovery in conjunction with current standard of care pharmaceuticals as well as when administered alone subsequent to surgical events or even as a prophylactic.

In addition to known glucose reduction attributes, the novel and hereby disclosed prophylactic glucose spike protective effects have substantial protective effects for optimizing metabolic interventions for cancer as well as surgical interventions for cancers including glioblastoma. With glioblastoma surgery in particular, the ability of bioidentical D-beta-hydroxybutyric acid to both guard against glucose spikes and reduce brain blood glucose in combination with substantial anti-seizure properties will provide substantial benefits in increasing successful outcomes for glioblastoma brain surgery and subsequent ‘push/pull’ anti-cancer metabolic therapies including 6-Diazo-5-oxo-1-norleucine (DON) which is a glutamine antagonist with robust anticancer efficacy and albendazole.

Free D-β-Hydroxybutyric Acid (D-BHB) functions as both a direct mitochondrial energy source and a powerful regulatory compound. Compared to synthetic esters and chemical salts, free D-BHB offers the fastest bioavailability and efficacy, delivering energy rapidly within minutes. It has demonstrated unexpected cognitive bioenergetic and neuroprotective benefits, making it unique among ketone forms. The free D-BHB format provides rapid release, is safe and non-toxic, and does not induce ketoacidosis. Historically used in human metabolism, it plays a central role in regulatory and direct mitochondrial energy processes.

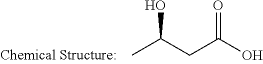

The rationale for a bioidentical D-BHB therapeutic regime is primarily supported by the neuroinflammation reduction properties of bioidentical D-BHB, high ketone levels generated by the same coupled with high bioavailability without salt load or liver metabolism requirements. As the bioidentical ‘free’ form. The bioidentical ‘free’ D-BHB form is immediately bioavailable for rapid metabolism which is materially distinct from the ‘ketone salt’ compounds (often D+L racemic) chemically joined to a salt or the synthetic ‘ketone ester’ compounds which are alcohol based and must be processed in the stomach and liver before releasing the ketone contained therein.

### BRIEF DESCRIPTION OF THE SEVERAL VIEWS OF THE DRAWING(S)

The accompanying drawings are included to better illustrate exemplary embodiments of the claimed invention.

FIG. 1 is a structural illustration of endogenous ketones.

FIG. 2 is a structural illustration of chemical structures of ketone compounds. FIG. 2A depicts bioidentical free D-beta-hydroxybutyric acid. FIG. 2B depicts a synthetic ketone ester, and FIG. 2C shows a chemical ketone salt.

FIG. 3 is a graphical illustration of endogenous ketone levels by age.

FIG. 4 is a graphical illustration of endogenous ketone brain metabolism.

FIG. 5 is a graphical illustration of comparative exogenous ketone blood levels.

FIG. 6 is a graphical illustration of anti-inflammatory metabolism of ketones.

FIG. 7 is a graphical illustration of free D-beta-hydroxybutyric acid preparation for protection and recovery.

FIG. 8 is a graphical illustration of free D-beta-hydroxybutyric acid preparation for spinal ischemia recovery.

FIG. 9 is a graphical illustration of D-BHB exogenous ketone blood levels.

FIG. 10 is a graphical illustration of D-BHB exogenous ketone blood levels.

FIG. 11 is a graphical illustration of continuous monitoring of body glucose levels.

FIG. 12 is a graphical illustration of continuous monitoring of body ketone levels resulting in improved deep sleep time due to enhanced D-BHB levels according to the claimed invention.

FIG. 13 is a schematic illustration of a preferred D-BHB sepsis management method with optional sepsis therapeutic pharmaceutical administration.

FIG. 14 is a schematic illustration of a preferred D-BHB sepsis management system with optional sepsis therapeutic pharmaceutical administration.

FIG. 15 illustrates an exemplary sepsis study design (1501).

FIG. 16 illustrates an illustrative embodiment of a sepsis recovery kit (1601).

FIG. 17 is a tabular illustration of baseline characteristics of enrolled participants by group.

FIG. 18 is a tabular illustration of mean acid-base and electrolyte parameters by treatment group during ICU follow-up.

FIG. 19 is a tabular illustration of individual mean acid-base and electrolyte results for each enrolled participant by treatment group.

FIG. 20 is a tabular illustration of mean follow-up severity scores by treatment group.

### DETAILED DESCRIPTION OF THE INVENTION

### Definitions

As used herein, “sepsis” refers to a life threatening organ dysfunction caused by a dysregulated host response to infection, for example as defined by Sepsis 3 criteria.

“Exogenous ketosis” refers to an increase in circulating ketone bodies, particularly beta hydroxybutyrate, achieved by administration of ketone compounds rather than by dietary or fasting interventions.

“Bioidentical free D βHB” denotes beta hydroxybutyrate in its D enantiomer form that is chemically identical to endogenous human D βHB and is formulated substantially free of added mineral salts and alcohol vehicles such as butanediol.

“Metabolic acidosis” broadly encompasses clinically relevant decreases in blood pH and bicarbonate with negative base excess that are not attributable to respiratory alkalosis or altitude related baseline differences, as determined by standard arterial blood gas analysis and clinical assessment.

### Exemplary Clinical Method

In one embodiment, adult patients diagnosed with sepsis according to Sepsis 3 criteria and admitted to an intensive care unit are screened for eligibility, excluding subjects with non sepsis related metabolic acidosis, pregnancy, or inability to receive enteral nutrition. Eligible patients are enrolled following informed consent and randomized in approximately a 1:1 ratio to receive either bioidentical free D βHB or a matching placebo, while all participants continue to receive standard sepsis management including nutritional support as prescribed by treating physicians. The active intervention comprises daily administration of about 10 g of bioidentical free D βHB for a period of about seven consecutive days, delivered orally or via nasogastric tube depending on clinical status.

In some embodiments, the patient population includes individuals with septic shock as defined by the Sepsis-3 criteria, comprising a vasopressor requirement to maintain mean arterial pressure of at least 65 mmHg and a serum lactate concentration greater than 2 mmol/L despite adequate volume resuscitation. In preferred embodiments, the patient is an adult admitted to an intensive care unit. Administration of bioidentical D-beta-hydroxybutyric acid is provided as an adjunct to standard-of-care sepsis management, which comprises antibiotic therapy, intravenous fluid resuscitation, and vasopressor support as indicated by the treating clinician; the D-beta-hydroxybutyric acid supplementation does not replace any element of established sepsis management.

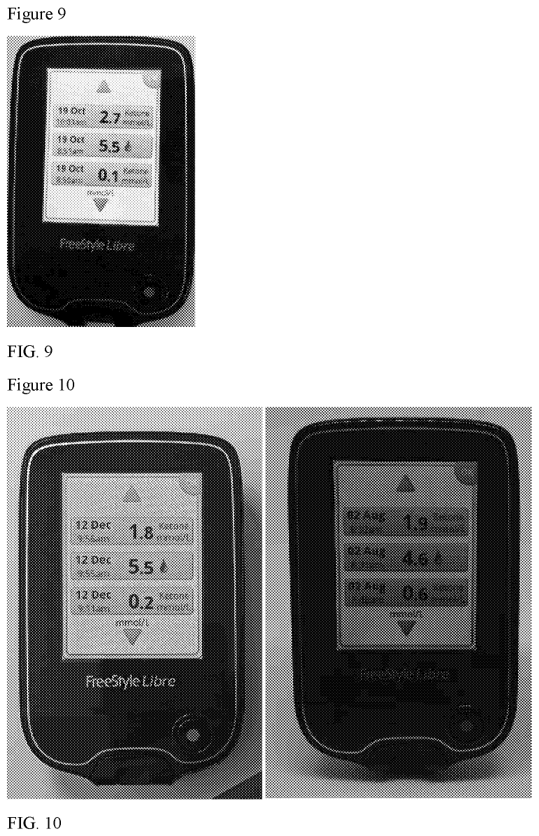

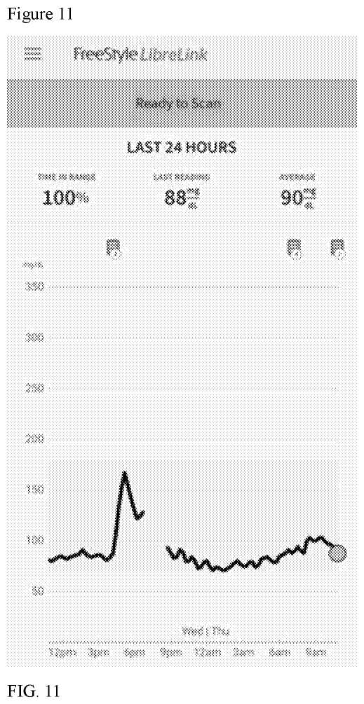

In preferred embodiments, the bioidentical D-beta-hydroxybutyric acid composition consists essentially of free D-beta-hydroxybutyric acid and pharmaceutically acceptable excipients and is substantially free of added mineral salts and alcohol vehicles. Administration is preferably initiated within 48 hours of establishing the diagnosis of sepsis, and the dosing regimen is selected to raise circulating D-beta-hydroxybutyrate to at least 0.3 mmol/L, with a preferred target of at least 0.5 mmol/L during the treatment period, as supported by the pharmacokinetic data described in FIGS. 9 and 10 of the parent application.

Placebo subjects receive an equivalent volume of water containing flavouring agents, such as citric flavour, to preserve blinding; the timing of each administration is coordinated to occur approximately one hour before routine arterial blood gas analysis to maximize the likelihood of detecting any supplementation related abnormalities.

### Monitoring and Outcomes

The primary safety endpoint is the incidence of supplementation attributable metabolic acidosis, defined by prespecified changes in arterial pH and base excess and assessed in conjunction with the clinical team to determine relatedness to D βHB administration. Secondary outcomes include group wise and individual changes in acid-base parameters (pH, pCO2, bicarbonate), serum electrolytes (including sodium, potassium, and chloride), and severity scores such as APACHE II, SAPS III, and SOFA over the follow up period. Blood samples are processed in accordance with standardized laboratory procedures, and interpretation accounts for local physiological conditions such as high altitude; for example, in Mexico City, lower pCO2 and bicarbonate levels are expected as normal, and values suggestive of respiratory alkalosis at sea level may be normal at altitude. Safety monitoring is continuous, and stopping rules may include: a decrease in arterial pH of at least about 0.05 units attributable to D βHB, refractory hypoglycaemia requiring intervention, severe metabolic acidosis with base excess less than about −12 mEq/L potentially related to the intervention, or withdrawal of consent.

In some embodiments, continued administration of bioidentical D-beta-hydroxybutyric acid is permitted so long as arterial pH remains within the range of 7.32 to 7.50 and no new electrolyte supplementation attributable to derangements outside the local laboratory reference range is required. These boundary conditions define a safety corridor within which the benefits of sustained exogenous ketosis may be pursued without unacceptable acid-base or electrolyte risk in the critically ill patient.

### Representative Results

In a representative implementation, nineteen adult sepsis patients are randomized, with nine receiving D βHB and ten receiving placebo, and all completing the study protocol with an average of approximately four acid-base measurements per patient during intensive care follow up. Across the follow up period, no cases of metabolic acidosis attributable to D βHB supplementation are identified, and no individual exhibits out of range bicarbonate values beyond the expected high altitude reference range.

Group level mean arterial pH, pCO2, and bicarbonate values remain similar between the D βHB and placebo groups, with mean pH around 7.38-7.40, pCO2 in the low 30 mmHg range, and bicarbonate around 19-20 mEq/L, and none of these parameters show statistically significant between group differences after correction for multiple comparisons.

Serum sodium, potassium, and chloride also remain comparable between the groups throughout follow up, with mean sodium around 139-140 mEq/L, potassium around 3.6-3.9 mEq/L, and chloride around 100-102 mEq/L, again without statistically significant differences.

Similarly, final severity scores including APACHE II, SAPS III, and SOFA do not differ significantly between the D βHB and placebo groups in this pilot cohort; however, the study is not powered for efficacy outcomes and these results should not be interpreted as demonstrating lack of therapeutic benefit.

The absence of supplementation related metabolic acidosis or electrolyte disturbance under the described dosing regimen supports the safety and tolerability of bioidentical free D βHB in sepsis and enables further development of exogenous ketosis as an adjunctive therapy.

### Mechanistic and Formulation Considerations

The biochemical rationale underlying the observed safety includes the recognition that acetoacetate and beta hydroxybutyrate are produced as conjugate bases, and that protons driving ketoacidosis originate from upstream metabolic processes such as lipolysis and fatty acyl CoA synthesis, which are bypassed by direct βHB supplementation.

Use of a salt free, alcohol free, bioidentical D βHB formulation minimizes additional mineral and alcohol load, which is particularly advantageous in critically ill patients with frequent kidney and liver dysfunction. In some embodiments, the D βHB formulation is provided as a ready to use oral or enteral solution with defined concentration (for example, about 10 g per unit dose), optional flavouring to improve palatability, and excipients compatible with enteral administration in critical care settings.

Alternative dosing regimens, including adjusted daily doses, extended treatment duration, or titration based on measured ketone levels, may be employed while maintaining monitoring principles and safety thresholds as described herein.

In some embodiments, the therapeutically effective daily amount of bioidentical D-beta-hydroxybutyric acid is expressed as a daily mass D in grams per day, selected to maintain a circulating D-beta-hydroxybutyrate concentration of at least 0.3 mmol/L during the treatment period. The daily mass D is determined according to the relation D=k×W, where W is the patient's body weight in kilograms and k is a dosing coefficient expressed in grams per kilogram per day. In various embodiments, k falls within the range of 0.1 g/kg/day to 1 g/kg/day, optionally adjusted over time based on laboratory measurements and clinical response. In preferred embodiments, k is between 0.25 g/kg/day and 1.0 g/kg/day.

In some embodiments, the calculated daily mass D is divided into two to six individual administrations distributed over a 24-hour period, the schedule being designed to be compatible with routine ICU medication rounds and blood draw schedules. In a preferred embodiment, each individual administration of bioidentical D-beta-hydroxybutyric acid is timed to occur between 30 and 120 minutes before a scheduled arterial blood gas analysis or routine laboratory blood draw.

In a safety-focused regimen, the daily mass D is approximately 10 g of bioidentical D-beta-hydroxybutyric acid administered for a period of 5 to 10 consecutive days. This regimen reflects the protocol employed in the randomized controlled clinical trial described herein, in which seven consecutive days of administration produced no supplementation-attributable metabolic acidosis or clinically significant electrolyte abnormalities. The range of 5 to 10 days accommodates variation in ICU length of stay and clinical recovery trajectories while maintaining the safety profile established by the trial data.

In some embodiments, one or more metabolic or inflammatory laboratory indicators are obtained before or during the treatment period and used to inform adjustment of the dosing coefficient k within the range of 0.1 g/kg/day to 1 g/kg/day. Indicators suitable for this purpose include C-reactive protein (CRP), the triglyceride to high-density lipoprotein cholesterol ratio (TG/HDL), glycated haemoglobin (HbA1c), total leukocyte count, absolute neutrophil count (ANC), and absolute lymphocyte count (ALC). These indicators collectively characterize the patient's inflammatory burden, metabolic reserve, and immune activation profile, each of which may influence the therapeutic window for exogenous ketosis in the sepsis setting.

The dosing coefficient k is increased toward an upper portion of the range-reflecting a higher daily dose of bioidentical D-beta-hydroxybutyric acid-when the patient exhibits a high inflammatory or dysmetabolic profile, defined by at least one of the following: CRP greater than 60 mg/L, indicating significant systemic inflammation; a TG/HDL ratio greater than 3.0, reflecting insulin resistance and impaired lipid metabolism; or HbA1c of 6.5% or higher, indicating pre-existing or concurrent glycaemic dysregulation. In such patients, the anti-inflammatory and bioenergetic benefits of D-beta-hydroxybutyric acid are expected to be proportionally greater, supporting a more aggressive dosing approach subject to ongoing safety monitoring.

Conversely, k is decreased toward a lower portion of the range when the patient exhibits increased risk of metabolic decompensation, indicated by at least one of the following: CRP greater than 150 mg/L, suggesting an extreme inflammatory state that may impair metabolic handling; a TG/HDL ratio greater than 6.0; HbA1c greater than 9.0%, reflecting severe chronic glycaemic dysregulation; total leukocyte count less than 4,000 cells/μL; or absolute neutrophil count less than 1,700 cells/μL. Leukopenia and neutropenia in this context suggest immune exhaustion or bone marrow suppression that may heighten metabolic vulnerability, warranting a conservative dosing posture.

In some embodiments, at least one of CRP, TG/HDL ratio, total leukocyte count, ANC, and ALC is re-measured at intervals of 24 to 72 hours during the treatment period, and k is dynamically adjusted based on observed changes in these values. The relevant immune activation profile may be characterized at baseline by leukocytosis, leukopenia, neutropenia, or lymphopenia, each of which carries distinct prognostic and dosing implications. In a preferred embodiment, a patient exhibiting neutrophilia with relative lymphopenia—defined as an ANC greater than 7,500 cells/μL in conjunction with an ALC less than 900 cells/μL—receives a minimum dosing coefficient k to be determined in future studies, reflecting the expectation that a hyperinflammatory immune phenotype will benefit from a higher ketogenic substrate load. A patient exhibiting leukopenia or neutropenia receives a maximum dosing coefficient k to be determined in future studies to reduce metabolic burden in the setting of immune compromise.

The claimed invention protects against sepsis and enhances hospital recovery by way of two primary embodiments. The first primary embodiment addresses sepsis by reducing inflammation and increasing direct mitochondrial energy by exogenously administering bioidentical D-beta-hydroxybutyric acid. The second primary embodiment improves existing sepsis therapeutic pharmaceutical interventions by co-administration of D-beta-hydroxybutyric acid to optimize sepsis therapeutic pharmaceutical compounds.

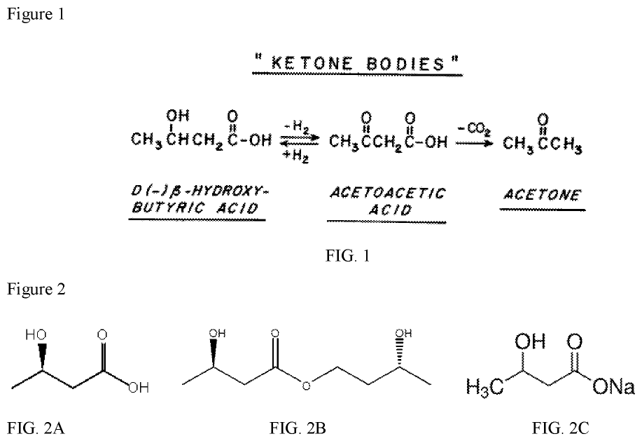

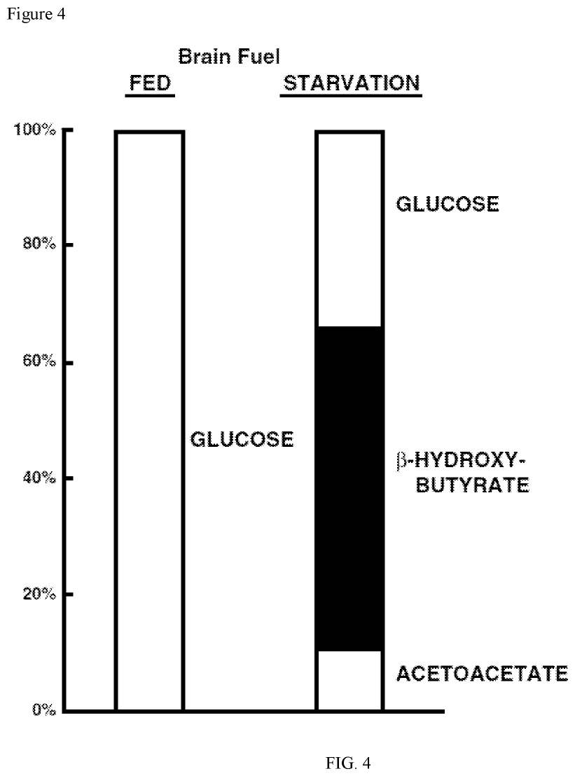

I. D-Beta HydroxyButyric Acid Compound Disclosure and Sepsis applications. FIG. 1 is a structural illustration of endogenous ketones. The illustration of the endogenously produced ketone bodies is from FIG. 4 of the Harvard Medical School Professor/Dr. George F. Cahill, Jr.'s lecture entitled “Ketosis” given over fifty years ago. Dr. Cahill's foundational work provides clear and concise understanding of ketosis to aid in the understanding of the claimed invention. The FIG. 2(a) chemical formula identifies the claimed bioidentical ‘free’ form of the exogenous D-beta-hydroxybutyric acid administered in the absence of a chemical salt or ester and is the subject of ClinicalTrials.gov identifier NCT05584371. The claimed exogenously administered bioidentical free D-beta-hydroxybutyric acid is chemically identical to the D-beta-hydroxybutyric acid identified in FIG. 1. Greater detail is hereby provided in the submitted BMJ Nutrition, Prevention & Health journal article. To be clear, this publication is the first reported clinical trial of bioidentical free D-beta-hydroxybutyric acid administered exogenously. The intervening fifty years between the Jeremiah Metzger Lecture given in Boston in 1972 and the present clinical trial and disclosure do not detail bioidentical free D-beta-hydroxybutyric acid but instead disclose a wide variety of precursors, chemical combinations and not naturally occurring synthetic products which are not bioidentical in their administered form.

FIG. 2 is a structural illustration of bioidentical ketone and chemically synthetic chemical ketone structures. FIG. 2A depicts bioidentical free D-beta-hydroxybutyric acid. FIG. 2B depicts the synthetic ketone ester and FIG. 2C shows the chemical ketone salt. Put simply, bioidentical free D-beta-hydroxybutyric acid offers direct mitochondrial energy resulting in optimized therapeutic outcomes when administered in the absence of a chemical salt or ester configuration. To date, exogenous ketone supplementation has been attempted as an aide in following the ketogenic diet. Unfortunately, ketone salts result in an abnormally high salt intake rate and the newly created (R)-3-hydroxybutyl (R)-3-hydroxybutyrate (D-β-hydroxybutyrate ester) or ‘ketone ester’ is a never before seen in man synthetic creation with bioavailability and other long-term concerns. FIG. 2A illustration of the chemical form of bioidentical free D-beta-hydroxybutyric acid is also distinguishable from ketone salts and ketone esters owing to its immediate bioavailability and absence of chemically joined salt or ester bond.

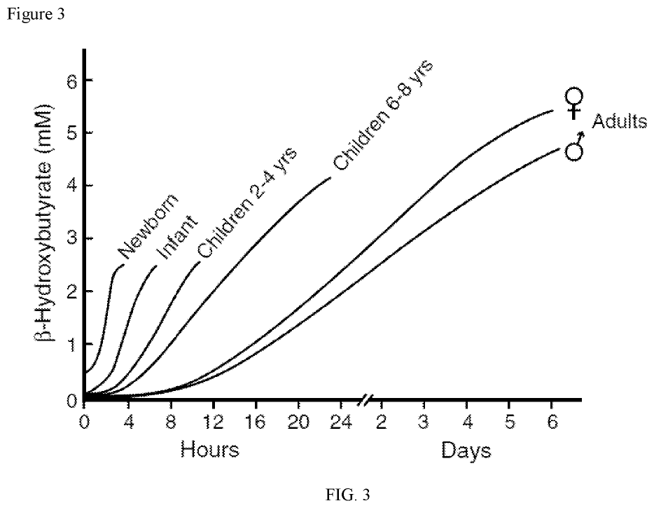

FIG. 3 is a graphical illustration of endogenous ketone levels by age and FIG. 4 is a graphical illustration of endogenous ketone brain metabolism from Dr./Prof. Cahill's journal article “Fuel Metabolism in Starvation” provided by Information Disclosure Statement. FIG. 3 details the time to ketosis in healthy adults vs children measured in hours or days. D-beta-hydroxybutyric acid is a naturally occurring ketone which is produced by the human body. The ketone plays a crucial role during development and throughout the healthy human lifespan. It is produced naturally during fasting (detailed by Cahill's graph above) as well as through the use of ketogenic diets. As Cahill observed, “Due to its use by brain, D-β-HydroxyButyric acid not only has permitted man to survive prolonged starvation, but also may have therapeutic potential owing to its greater efficiency in providing cellular energy in ischemic states such as stroke, myocardial insufficiency, neonatal stress, genetic mitochondrial problems, and physical fatigue.” Cahill's early insights on D-β-hydroxybutyric acid are now through Applicant's clinical trial efforts becoming proven in reality. Applicant's clinical trials including ClinicalTrials.gov identifier NCT05584371 are the first documented clinical applications of free D-beta-hydroxybutyric acid as further detailed in the provided Dr.Prof. Soto-Mota IDS journal article. FIG. 4 details the fact that ketones in general, and D-beta-hydroxybutyric acid in particular is the preferred fuel in the human brain. The time to ketosis levels are relevant in the context of the instant application in that creating a rapid 1.5-2.5 mmol/L increase in ketone levels is simply not practical through fasting or diet when rapidly needed in a sepsis protection or hospital recovery context.

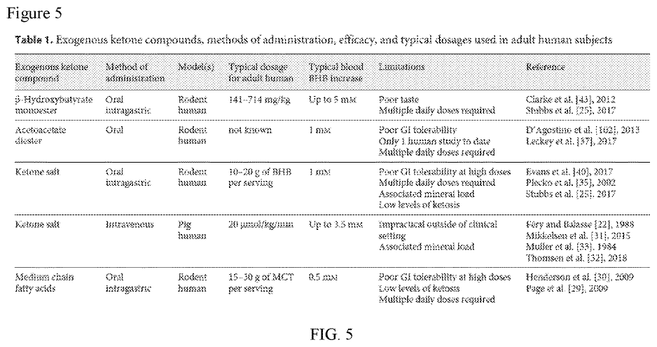

FIG. 5 is a graphical illustration of comparative exogenous ketone blood levels. FIG. 5 depicts Table 1 from “Exogenous Ketone Bodies as Promising Neuroprotective Agents for Developmental Brain Injury” and details the significant limitations among the variety of previously known exogenous salt and ester ketone compounds. Ketone salts are often racemic D+L mixtures which rarely provide greater than 1 mmol/L ketone increase along with substantial salt load rendering it unsuitable for long term daily administration. Similarly, while the synthetic ‘ketone ester’ does provide a higher ketone level response, its highly unpalatable taste renders it highly unsuitable for use in mental health applications. FIG. 5 does not include free D-beta-hydroxybutyric acid due to the fact that ClinicalTrials.gov identifier NCT05584371 and the provided BMJ Nutrition, Prevention & Health journal article is the first instance of exogenous free D-beta-hydroxybutyric acid journal publication. Put simply, there has been a longstanding need for a salt free, bioidentical solution to rapid ketosis without alcohol or need for further liver metabolism to rapidly increase ketones to a level relevant for sepsis protection and hospital recovery.

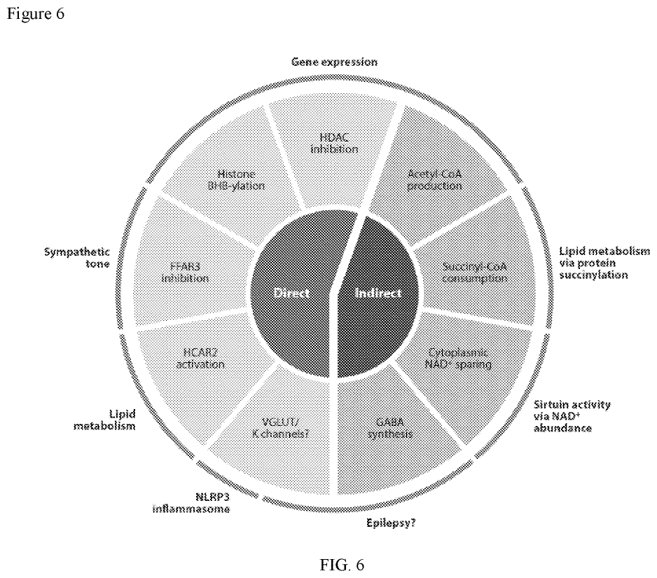

FIG. 6 is a graphical illustration of brain metabolism of ketones from Prof Newman and Prof. Verdin's journal article “β-Hydroxybutyrate: A Signaling Metabolite” figure number 2. This figure details “Schematic of direct and indirect signaling functions of the ketone body βHB. Indirect signaling functions require catabolism to other molecules, whereas direct signaling functions are actions of βHB itself . . . ” Applicants respectfully submits that Professors Newman and Verdin detail the starting point of the anti-inflammatory nature of D-beta-hydroxybutyric acid. As supplementation with bioidentical free D-beta-hydroxybutyric acid is anti-inflammatory it is logical to conclude that reduction of sepsis related events will follow. It is a direct and foreseeable consequence of the claimed invention that similar benefits for recovery from cardiac conditions, stroke and spinal ischemia are achieved. In terms of cardiac applications, Applicant hereby provides as IDS reference 7 the article entitled “Ketone Body β-Hydroxybutyrate Prevents Myocardial Oxidative Stress in Septic Cardiomyopathy” which states “In this study, the ketone body β-OHB was shown to prevent LPS-induced myocardial injury and cardiac dysfunction. Mechanistically, as an HDAC inhibitor, β-OHB activated the antioxidant FoxO3a/MT2 pathway. This response enhanced redox homeostasis and improved mitochondrial function. These findings, therefore, suggest that β-OHB is an effective agent for myocardial protection in sepsis.” (emphasis added) While the study is limited to mouse model applications and utilized the synthetic ketone ester, it is reasonable to conclude that for human applications the bioidentical form of free D-beta-hydroxybutyric acid will yield superior cardiac protection against sepsis owning to its immediate bioavailability and lack of an alcohol chemical moiety.

Applicant provides by way of IDS5 the article entitled “The ketone metabolite β-hydroxybutyrate blocks NLRP3 inflammasome-mediated inflammatory disease” as similar support for a broader range of anti-inflammatory and consequently reduced sepsis and enhanced hospital recovery applications. The article by Youm states in relevant part on page 6, “These findings suggest that the fasting- or exercise-induced metabolite βHB inhibits the NLRP3 inflammasome in macrophages independently of binding to surface GPR109A receptors or mitochondrial oxidation, which may avoid both competition for receptor occupancy and requirement for ATP generation” and notes that “in states of extreme energy deficit such as starvation, metabolic signals such as βHB can dampen innate immune responses, sparing ATP for the functioning of ketone-dependent organs such as the brain and heart” which holds promise in reducing the “severity of NLRP3-mediated chronic inflammatory diseases.” Consequently, providing bioidentical free D-beta-hydroxybutyric acid according to the claimed invention should not only reduce incidences of sepsis but also promote enhanced hospital recovery as well.

In terms of sepsis mitigation therapeutic compounds incorporating free D-beta-hydroxybutyric acid, while preferred embodiments are provided as 5 g-30 g format, larger and smaller amounts may be provided while maintaining the spirit and scope of the invention. In a preferred embodiment, free D-beta-hydroxybutyric acid is combined and provided in conjunction with a ketogenic complementary nutritional supplement. In an illustrative embodiment, the ketogenic complementary nutritional supplement is selected from arginine, omega-3 fatty acids, and Vitamin D. There is a wide variety of choices for ketogenic complementary supplements which can include precursors such as Medium Chain Triglycerides, plant oils such as olive oil, coconut oil, caprylic acid and other standard ingredients in a ketogenic diet. The primary component to be avoided is any kind of sugar, whether in the form of sucrose, glucose, fructose or the like. Additionally, an alternate preferred embodiment includes a palatability agent incorporating sugar free sweetener such as stevia or allulose as well as natural fruit essence distillate as a palatability enhancer. In an alternate preferred embodiment, one or more sepsis therapeutic pharmaceuticals are added including antibiotics, vasopressors, anticoagulants, corticosteroids and immunoglobulins administered in a physiologically therapeutic amount.

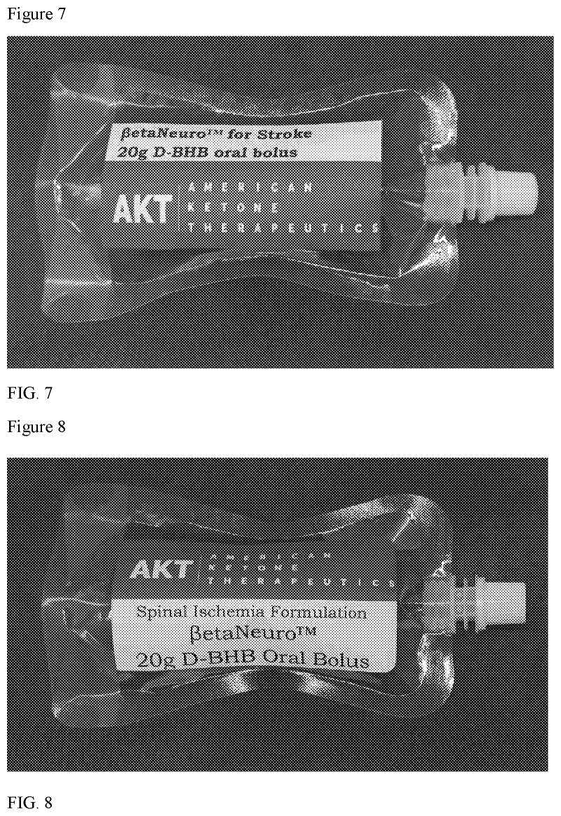

FIG. 7 is a graphical depiction of an illustrative example of free D-beta-hydroxybutyric acid preparation for hospital recovery. The depicted preparation of D-βHB free acid is designed for rapid and easy administration for hospital recovery and may alternately be utilized for sepsis protection. While 20 g D-βHB free acid is depicted a wide range of D-βHB administration is anticipated according to the claimed invention. FIG. 8 is a graphical illustration of free D-beta-hydroxybutyric acid preparation for spinal ischemia recovery. The depicted preparation of D-βHB free acid is designed for rapid and easy intubated administration. While 20 g D-βHB free acid is depicted a wide range of D-βHB administration in grams is anticipated according to the claimed invention. In addition to the aforementioned bioenergetic properties, long term administration of D-βHB free acid improves spinal ischemia prognosis owing to the enhanced atherosclerosis attributes even in the absence of a co-administered pharmaceutical. It is an anticipated embodiment of the claimed invention that in addition to exigent D-βHB administration, follow-on D-βHB administration between 5 g and 30 g regularly administered over days, weeks, months and even years can improve brain stroke and spinal ischemia positive outcomes and enhance recovery.

FIG. 9 is a graphical illustration of D-βHB exogenous ketone blood levels. The above Abbot blood ketone meter evidenced by Abbott blood test strip depicts the rapid rise in blood ketone levels from 0.1 mmol/L (absence of ketosis) to 2.7 mmol/L (ketosis) after oral bolus administration of 15 grams of bioidentical free D-beta-hydroxybutyric acid. The ketone levels depicted are attained in the absence of a salt as well as without ester bonds. It is the free acid D-βHB exogenously administered which is bio-identical to the endogenous D-βHB free acid generated by the human body. The bioidentical ketone body D-βHB is first and foremost an energy source, which is directly and preferentially metabolized by the brain. D-βHB is also active as a signaling mechanism and neuro-inflammation reduction agent, resulting in broad and beneficial brain bioenergy related benefits.

FIG. 10 is a graphical illustration of D-βHB exogenous ketone blood levels. FIG. 10 details rapid ketosis within 45 minutes confirmed by Abbott blood test strip after 10 g D-βHB with palatability agent administered by oral drink. Blood ketone levels document rise from 0.2 mmol/L to 1.8 mmol/L and 0.6 mmol/L to 1.9 mmol/L in a variety of test subjects. FIG. 10 reinforces the fact that supplementation by utilizing free D only beta-hydroxybutyric acid without ketone salt or ketone ester is a reliable, reproduceable method and system for rapid ketone level increase for stroke recovery and maintenance. While amounts of free D only beta-hydroxybutyric acid taken for stroke effects may vary, it is an anticipated preferred embodiment that levels between 5 and 30 grams of D only beta-hydroxybutyric acid are taken as needed for optimum recovery. Alternate embodiments may include lower or higher amounts, however, experimental evidence has confirmed optimal administration in this range for a wide variety of other applications. As demonstrated in the parent application, oral administration of 10 g of bioidentical D-beta-hydroxybutyric acid reliably raises circulating D-beta-hydroxybutyrate concentrations to between 1.8 and 1.9 mmol/L within 45 minutes, well above the 0.5 mmol/L preferred threshold and the 0.3 mmol/L minimum threshold recited in the claims herein.

FIG. 11 is a graphical illustration of continuous monitoring of body glucose levels. FIG. 11 depicts continuous glucose monitoring levels with carbohydrate challenge documented by Abbot Libre Continuous Glucose Monitoring (CGM) device. In this illustrative embodiment, glucose levels from a 50 g carbohydrate ingestion are depicted unaided by free D-beta-hydroxybutyric acid on the left and repeated the following day with two 50 g carbohydrate ingestions accompanied by 10 g free D-beta-hydroxybutyric acid administered 45 minutes before the carbohydrate insult. The data is noteworthy in that while the equivalent amount of identical carbohydrates were consumed, the rapid glucose spike depicted in the unsupplemented ingestion is not found with 10 g D-BetaHyrdoxyButyric Acid accompaniment. This data is relevant in the stroke context owing to the fact that certain individuals suffering from brain energy deficits often report higher susceptibility due to low ketones and high glucose. As free D-beta-hydroxybutyric acid can lower glucose while rapidly increasing ketone levels, it is believed that obtaining high ketones while lowering glucose will yield additional stroke recovery benefits for high glucose sensitive individuals suffering from sepsis and may be considered in lieu of insulin administration.

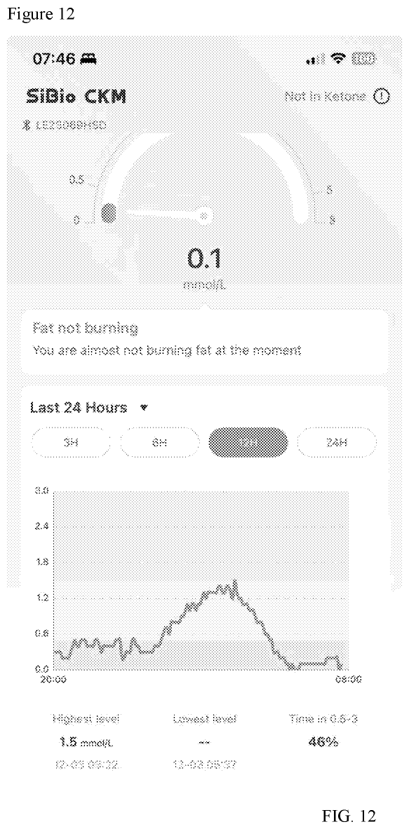

FIG. 12 is a graphical illustration of continuous monitoring of body ketone levels resulting in improved deep sleep time due to enhanced D-βHB levels according to the claimed invention. It is noteworthy that consumption of even small amounts of free D-beta-hydroxybutyric acid can give rise to therapeutic ketosis levels as measured by Continuous Ketone Meter (CKM). In this representative example, 5 grams of free D-beta-hydroxybutyric acid consumed 45 minutes prior to bedtime resulted in a 1.5 mmol/L increase can also enhance the quantity of deep sleep obtained. While free D-beta-hydroxybutyric acid is not a sedative, even small amounts can greatly improve deep sleep quantity and quality. It is an intended and foreseeable embodiment application to increase deep sleep with free D-beta-hydroxybutyric acid with the aim of guarding against sepsis, improving hospital recovery and enhanced resiliency.

### II. D-Beta HydroxyButyric Acid Sepsis Recovery Method & System Disclosure and Applications for Pharmaceutical Co-Administration

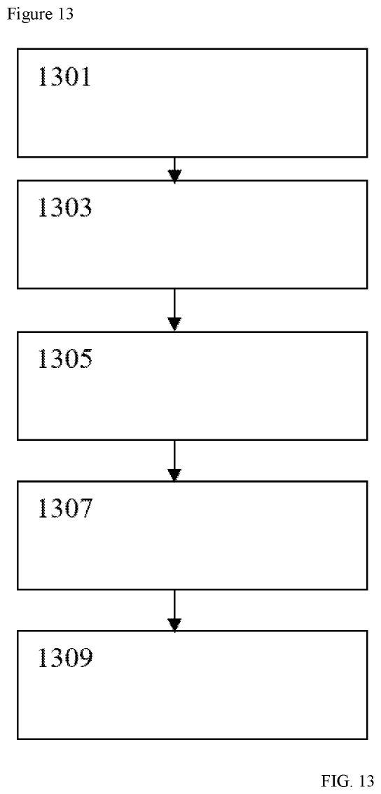

FIG. 13 is a schematic illustration of a preferred bioidentical D-beta-hydroxybutyric acid sepsis protection/hospital recovery management method with optional pharmaceutical administration. In a preferred embodiment, a method of sepsis symptom management is obtained through the steps of:
Assessing (1301) ketone levels and sepsis symptoms,
Administering (1303) a therapeutic amount of free D-beta-hydroxybutyric acid,
Optionally dosing (1305) a therapeutic amount of sepsis therapeutic pharmaceutical,
Evaluating (1307) the sepsis symptoms of a subject in need, and
Readministering (1309) free D-beta-hydroxybutyric acid alone or in combination with optionally redosing together with a therapeutic amount of sepsis therapeutic pharmaceutical.

In terms of sepsis therapeutic pharmaceuticals, known approaches to treat sepsis include antibiotics, vasopressors, intravenous (IV) fluids, anticoagulants, corticosteroids, insulin and immunoglobulins. Broad-spectrum antibiotics are often the first line of treatment, administered as soon as sepsis is suspected. Once the specific bacteria causing the infection is identified, more targeted antibiotics are used. Vasopressors are used to raise blood pressure in septic shock, a severe condition associated with sepsis where blood pressure drops dangerously low. Common vasopressors include norepinephrine, dopamine, and vasopressin. Intravenous (IV) Fluids are used for fluid resuscitation which is crucial to maintain adequate blood pressure and organ perfusion of which saline or lactated Ringer's solution are commonly used. Anticoagulants are used to prevent or treat disseminated intravascular coagulation (DIC), a complication of sepsis. Anticoagulants include Heparin which is a commonly used anticoagulant for sepsis. Corticosteroids are used in severe cases of sepsis or septic shock, especially when the response to IV fluids and vasopressors is inadequate to reduce inflammation. Insulin Therapy is used to control high blood sugar levels, which can accompany severe sepsis or septic shock. Immunoglobulins may also be used in severe sepsis cases.

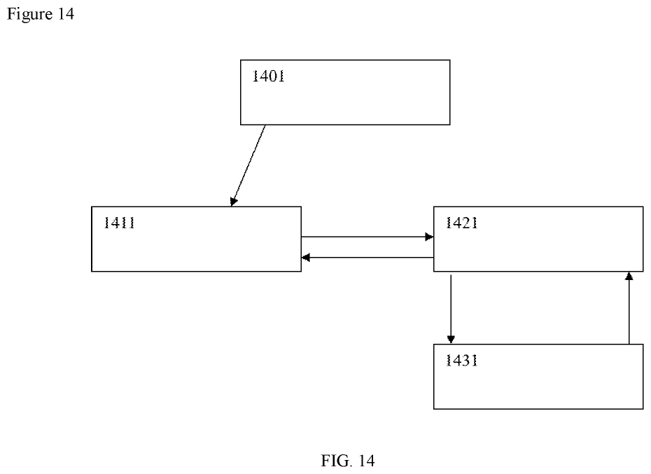

FIG. 14 is a schematic illustration of a preferred D-βHB sepsis management system with optional sepsis therapeutic pharmaceutical administration. In the illustrative embodiment, ketone level monitor (1421) determines the amount of free D-beta-hydroxybutyric acid for a sepsis subject in need thereof, optionally and additionally informed by glucose monitor (1433). Free D-beta-hydroxybutyric acid therapeutic dose (1411) is administered alone or in conjunction with optional sepsis therapeutic pharmaceutical therapeutic dose (1401).

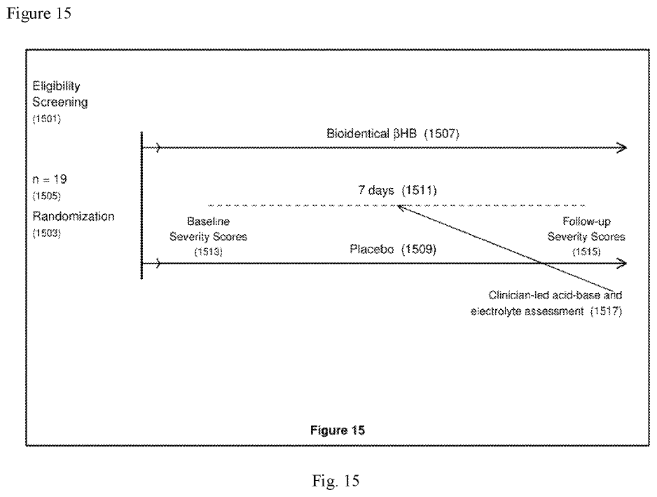

FIG. 15 illustrates an exemplary study design for evaluating the safety and tolerability of exogenous ketosis induced by bioidentical D-βHB in patients with sepsis, showing eligibility screening (1501), randomization (1503) of nineteen enrolled patients (1505) into a bioidentical D-βHB arm (1507) and a placebo arm (1509), a seven-day intervention period (1511), baseline severity scores (1513) and follow-up severity scores (1515) assessed at the start and end of the intervention period respectively, and a clinician-led acid-base and electrolyte assessment (1517) performed throughout the treatment period.

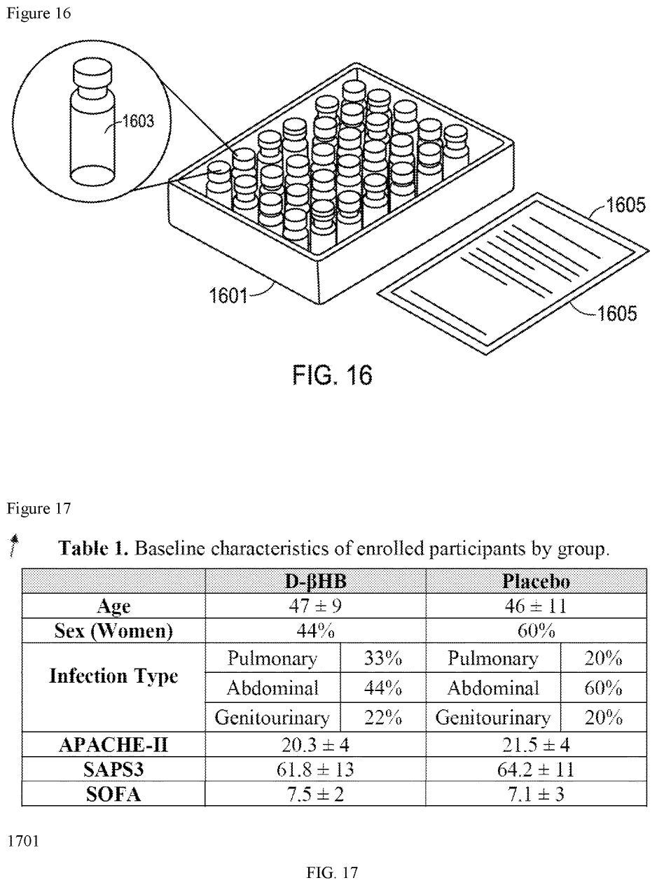

FIG. 16 illustrates an embodiment as an administration kit (1601) for inducing exogenous ketosis in a human patient with sepsis. The kit comprises a plurality of unit dose containers (1603), each containing a salt-free, alcohol-free, ester-free bioidentical D-beta-hydroxybutyric acid composition in an amount suitable for a single enteral administration via oral or nasogastric route. In preferred embodiments, each unit dose contains between 5 g and 20 g of net bioidentical D-beta-hydroxybutyric acid. The kit further comprises printed or electronic instructions for use (1605) directed to a clinician, specifying that unit doses are to be administered to raise blood D-beta-hydroxybutyrate to a ketotic range of at least 0.3 mmol/L, with administration initiated within 48 hours of establishing the diagnosis of sepsis. In a preferred embodiment, the instructions describe at least two regimens: a safety-focused regimen comprising approximately 10 g per day for 5 to 10 consecutive days; and at least one higher-dose regimen defined by the weight-based dosing coefficient k in the range of 0.25 g/kg/day to 1.0 g/kg/day of net bioidentical D-beta-hydroxybutyric acid, with guidance for adjustment based on clinical and laboratory response as described herein.

FIG. 17 presents baseline characteristics of the nineteen enrolled participants stratified by treatment group (1701). In the D-βHB group (n=9), mean age was 47±9 years, 44% were women, and infection sources comprised pulmonary (33%), abdominal (44%), and genitourinary (22%) etiologies, with baseline severity scores of APACHE-II 20.3±4, SAPS III 61.8±13, and SOFA 7.5±2. In the placebo group (n=10), mean age was 46±11 years, 60% were women, and infection sources comprised pulmonary (20%), abdominal (60%), and genitourinary (20%) etiologies, with baseline severity scores of APACHE-II 21.5±4, SAPS III 64.2±11, and SOFA 7.1±3. The comparability of baseline severity scores between groups confirms adequate randomization and supports interpretation of follow-up data as reflecting treatment effects rather than baseline imbalance.

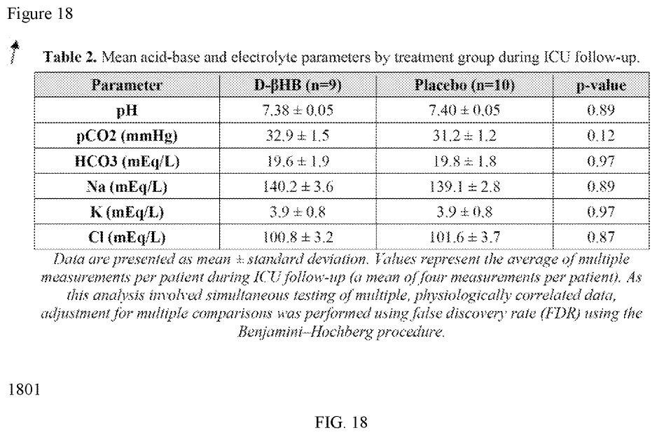

FIG. 18 presents mean acid-base and electrolyte parameters measured during ICU follow-up, representing the average of approximately four measurements per patient, with FDR adjustment for multiple comparisons using the Benjamini-Hochberg procedure (1801). In the D-βHB group (n=9), mean arterial pH was 7.38±0.05, pCO2 32.9±1.5 mmHg, HCO3 19.6±1.9 mEq/L, sodium 140.2±3.6 mEq/L, potassium 3.9±0.8 mEq/L, and chloride 100.8±3.2 mEq/L. In the placebo group (n=10), corresponding values were pH 7.40±0.05, pCO2 31.2±1.2 mmHg, HCO3 19.8±1.8 mEq/L, sodium 139.1±2.8 mEq/L, potassium 3.9±0.8 mEq/L, and chloride 101.6±3.7 mEq/L. No parameter showed a statistically significant between-group difference (all FDR-adjusted p-values≥0.12), confirming the absence of supplementation-attributable acid-base disturbance or clinically significant electrolyte derangement under the described dosing regimen.

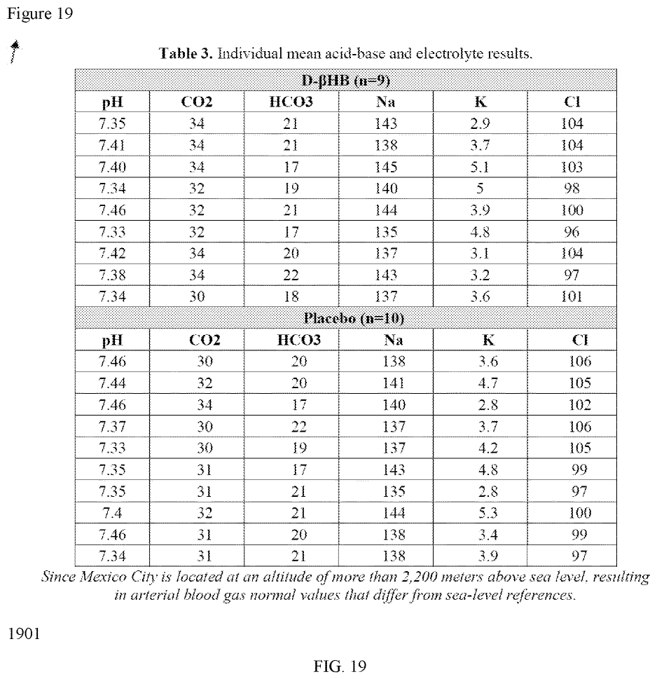

FIG. 19 presents individual patient-level mean acid-base and electrolyte values for each of the nineteen enrolled participants, stratified by treatment group (1901). In the D-βHB group (n=9), individual arterial pH values ranged from 7.33 to 7.46, pCO2 from 30 to 34 mmHg, HCO3 from 17 to 22 mEq/L, sodium from 135 to 145 mEq/L, potassium from 2.9 to 5.1 mEq/L, and chloride from 96 to 104 mEq/L. In the placebo group (n=10), individual arterial pH values ranged from 7.33 to 7.46, pCO2 from 30 to 34 mmHg, HCO3 from 17 to 22 mEq/L, sodium from 135 to 144 mEq/L, potassium from 2.8 to 5.3 mEq/L, and chloride from 97 to 106 mEq/L. Notably, no individual D-βHB-treated patient exhibited arterial pH outside the safety corridor of 7.32 to 7.50 described herein, and no individual required new electrolyte supplementation attributable to D-βHB administration. Values should be interpreted in the context of the Mexico City study site, located at more than 2,200 meters above sea level, where lower pCO2 and bicarbonate values reflect normal high-altitude physiology rather than pathological acid-base disturbance.

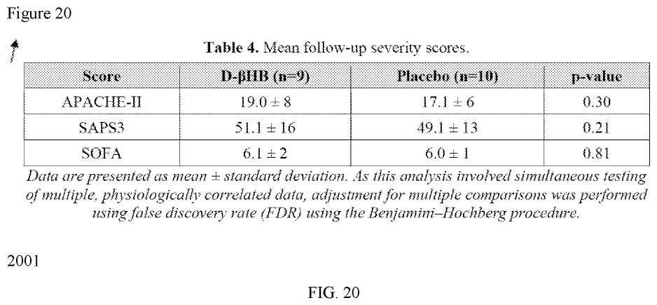

FIG. 20 presents mean follow-up severity scores for each treatment group (2001), with FDR adjustment for multiple comparisons using the Benjamini-Hochberg procedure. In the D-βHB group (n=9), mean follow-up APACHE-II was 19.0±8, SAPS III 51.1±16, and SOFA 6.1±2. In the placebo group (n=10), mean follow-up APACHE-II was 17.1±6, SAPS III 49.1=13, and SOFA 6.0±1. No severity score showed a statistically significant between-group difference (FDR-adjusted p-values 0.30, 0.21, and 0.81 for APACHE-II, SAPS III, and SOFA respectively). Consistent with the specification, the absence of significant between-group differences in follow-up severity scores reflects the limited statistical power of this pilot cohort rather than a conclusion of therapeutic equivalence, and these results do not preclude a clinically meaningful benefit of D-βHB administration on organ dysfunction progression as monitored under claim 19.

In the description, numerous specific details are set forth in order to provide a thorough understanding of the present embodiments. It will be apparent, however, to one having ordinary skill in the art that the specific detail need not be employed to practice the present embodiments. In other instances, well-known materials or methods have not been described in detail in order to avoid obscuring the present embodiments. In particular, free D-beta-hydroxybutyric acid may be administered diluted or with a preferred palatability agent without detracting from the spirit or scope of the claimed invention.

Reference throughout this specification to “one embodiment”, “an embodiment”, “one example” or “an example” means that a particular feature, structure or characteristic described in connection with the embodiment or example is included in at least one embodiment of the present embodiments. Thus, appearances of the phrases “in one embodiment”, “in an embodiment”, “one example” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

As used herein, the terms “comprises,” “comprising,” “includes,” “including,” “has,” “having,” or any other variation thereof, are intended to cover a non-exclusive inclusion. For example, a process, article, or apparatus that comprises a list of elements is not necessarily limited to only those elements but may include other elements not expressly listed or inherent to such process, article, or apparatus. Additionally, any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” and “in one embodiment.”

### Alternative Routes of Administration

While the preferred embodiments of the present invention are directed to enteral administration of bioidentical D-beta-hydroxybutyric acid-including oral and nasogastric routes—it is contemplated that the active agent may alternatively be administered by parenteral routes, including intravenous infusion, in critically ill patients who are unable to tolerate enteral nutrition due to conditions such as ileus, bowel obstruction, or post-surgical contraindications to enteral feeding. In such circumstances, a sterile aqueous solution of free D-beta-hydroxybutyric acid, substantially free of mineral salts and alcohol vehicles, may be formulated for intravenous infusion and administered at a daily dose determined according to the relation D=k×W, where W is the patient's body weight in kilograms and k is a dosing coefficient within the range of 0.1 g/kg/day to 1 g/kg/day, with the objective of raising circulating D-beta-hydroxybutyrate to a ketotic range of at least 0.3 mmol/L. The same safety monitoring parameters described herein-including arterial pH, pCO2, bicarbonate, and serum electrolytes- and the same predefined stopping criteria apply to parenteral administration. The inventors expressly contemplate parenteral administration as a variant within the scope of the disclosed invention, and this disclosure is intended to constitute prior art with respect to any third party claim to parenteral administration of free D-beta-hydroxybutyric acid in the treatment of sepsis filed subsequent to the priority date of this application.

We claim:

1. A method of treating a human patient with sepsis, the method comprising:
raising the patient's blood concentration of D-beta-hydroxybutyrate to a therapeutically effective ketotic range of at least 0.3 mmol/L by enteral administration of a salt-free, alcohol-free, ester-free bioidentical D-beta-hydroxybutyric acid composition.

2. The method of claim 1, wherein the patient has septic shock as defined by the Sepsis-3 criteria, comprising vasopressor requirement to maintain mean arterial pressure≥65 mmHg and serum lactate>2 mmol/L despite adequate volume resuscitation.

3. The method of claim 1, wherein the bioidentical D-beta-hydroxybutyric acid composition consists essentially of free D-beta-hydroxybutyric acid and pharmaceutically acceptable excipients and is substantially free of added mineral salts and alcohol vehicles; wherein the bioidentical D-beta-hydroxybutyric acid is administered as an adjunct to standard-of-care sepsis management comprising antibiotic therapy, intravenous fluid resuscitation, and vasopressor support as indicated; and wherein the patient is an adult admitted to an intensive care unit.

4. The method of claim 1, wherein the administration raises circulating D-beta-hydroxybutyrate to at least 0.5 mmol/L during the treatment period and the administration is initiated within 48 hours of establishing the diagnosis of sepsis.

5. The method of claim 1, wherein the therapeutically effective amount of bioidentical D-beta-hydroxybutyric acid is defined by a daily mass D (g/day) selected to achieve the desired ketotic range wherein D is determined to maintain circulating D-beta-hydroxybutyrate at a concentration of at least 0.3 mmol/L during the treatment period.

6. The method of claim 5, wherein the daily mass D is determined according to the relation D=k×W,
where W is the patient's body weight (kg), and k is a dosing coefficient between 0.1 g/kg/day and 1 g/kg/day of bioidentical D-beta-hydroxybutyric acid, optionally adjusted over time based on laboratory and clinical responses.

7. The method of claim 6, wherein k is between 0.25 g/kg/day and 1.0 g/kg/day.

8. The method of claim 6, wherein the daily dose of bioidentical D-beta-hydroxybutyric acid is divided into two to six administrations over 24 hours, as compatible with routine ICU medication and blood draw schedules.

9. The method of claim 6, wherein each administration of bioidentical D-beta-hydroxybutyric acid occurs 30 to 120 minutes before a scheduled arterial blood gas analysis or routine laboratory blood draw.

10. The method of claim 5, wherein, in a safety-focused regimen, the daily mass D is about 10 g of bioidentical D-beta-hydroxybutyric acid for a period of 5 to 10 consecutive days.

11. The method of claim 1, further comprising monitoring arterial pH, partial pressure of carbon dioxide, bicarbonate, and serum sodium, potassium, and chloride during the treatment period.

12. The method of claim 11, further comprising applying predefined stopping or dose reduction criteria of bioidentical D-beta-hydroxybutyric acid that include at least one of:
(a) a decrease in arterial pH of at least 0.05 units temporally associated with bioidentical D-beta-hydroxybutyric acid administration;
(b) refractory hypoglycaemia requiring intervention; or
(c) severe metabolic acidosis with base excess less than-12 mEq/L potentially related to the administration.

13. The method of claim 12, wherein continued administration of bioidentical D-beta-hydroxybutyric acid is permitted so long as arterial pH remains between 7.32 and 7.50 and no new supplementation attributable to electrolyte derangements outside the local reference range is detected.

14. The method of claim 6, further comprising, before or during treatment, obtaining one or more metabolic or inflammatory indicators selected from:
C-reactive protein (CRP), triglyceride to high-density lipoprotein cholesterol ratio (TG/HDL), glycated haemoglobin (HbA1c), total leukocyte count, absolute neutrophil count, and absolute lymphocyte count,
and adjusting the dosing coefficient k within the range from 0.1 g/kg/day to 1 g/kg/day of bioidentical D-beta-hydroxybutyric acid based at least in part on one or more indicators.

15. The method of claim 14, wherein k is increased toward an upper portion of the range when the patient exhibits a high inflammatory or dysmetabolic profile defined by at least one of:
(a) CRP greater than 60 mg/L;
(b) TG/HDL ratio greater than 3.0; or
(c) HbA1c of 6.5% or higher.

16. The method of claim 14, wherein k is decreased toward a lower portion of the range when the patient exhibits increased risk of metabolic decompensation defined by at least one of:
(a) CRP greater than 150 mg/L;
(b) TG/HDL ratio greater than 6.0;
(c) HbA1c greater than 9.0%;
(d) total leukocyte count less than 4,000 cells/μL; or
(e) absolute neutrophil count less than 1,700 cells/μL.

17. The method of claim 16, further comprising repeating measurement of at least one of CRP, TG/HDL ratio, total leukocyte count, absolute neutrophil count, and absolute lymphocyte count at intervals of 24 to 72 hours and dynamically adjusting k based on changes in the measured values, wherein the immune activation profile is characterized by baseline leukocytosis, leukopenia, neutropenia, or lymphopenia.

18. The method of claim 1, further comprising obtaining a baseline organ dysfunction score selected from SOFA, APACHE-II, and SAPS III prior to initiating administration of bioidentical D-beta-hydroxybutyric acid, and repeating measurement of the selected score at intervals of 24 to 72 hours during the treatment period to monitor for attenuation of organ dysfunction progression.

19. An administration kit for inducing exogenous ketosis in a human patient with sepsis, comprising:
(a) a plurality of unit doses of a salt-free, alcohol-free, ester-free bioidentical D-beta-hydroxybutyric acid composition suitable for enteral administration, including oral and nasogastric routes; and
(b) printed or electronic instructions for use that direct a clinician to administer the unit doses to raise blood D-beta-hydroxybutyrate into a ketotic range of at least 0.3 mmol/L within 48 hours of establishing a diagnosis of sepsis and wherein each unit dose contains between 5 g and 20 g of net bioidentical D-beta-hydroxybutyric acid.

20. The administration kit of claim 19, wherein the instructions describe:
a safety-focused regimen comprising about 10 g/day of bioidentical D-beta-hydroxybutyric acid for 5 to 10 days, and
at least one higher dose regimen defined by a dosing coefficient k between 0.25 g/kg/day and 1.0 g/kg/day of net bioidentical D-beta-hydroxybutyric acid.
